(19) United States
(12) Patent Application Publication
(10) Pub. No.: US 2017/0265483 A1
(43) Pub. Date: Sep. 21, 2017

(54) USE OF PENICILLIUM GLUCOSE OXIDASE IN THE BAKING INDUSTRY FOR REPLACING BROMATE OR AZODICARBONAMIDE

(71) Applicant: DSM IP ASSETS B.V., Heerlen (NL)

(72) Inventors: José DIAS PERES, Echt (NL); Caroline Hendrine Maria VAN BENSCHOP, Echt (NL); Soon Hee ONG, Echt (NL); Lambertus Gerhard STRUBBE, Echt (NL)

(21) Appl. No.: 15/505,080

(22) PCT Filed: Aug. 18, 2015
(86) PCT No.: PCT/EP2015/068911

§ 371 Date: Feb. 18, 2017

(30) Foreign Application Priority Data
Aug. 21, 2014 (CN) 1201410415260.5
Sep. 3, 2014 (EP) 14183378.0
Sep. 30, 2014 (CN) 201410522889.X
Oct. 6, 2014 (CN) 201410522369.9

(51) Int. Cl. A21D 2/22; A21D 10/00; A21D 8/04; C12N 9/04; C12N 9/34

(52) U.S. Cl. CPC A21D 2/22; C12Y 101/03004; C12Y 302/01003; C12N 9/0006; C12N 9/2428; A21D 8/042; A21D 10/002

(57) ABSTRACT

The present invention relates to the use of a Penicillium glucose oxidase as a chemical oxidizer replacement in the food industry.

### FIELD OF THE INVENTION

The present invention relates to application of glucose oxidase in the baking industry.

### BACKGROUND OF THE INVENTION

Glucose oxidases (GOX) catalyze the oxidation of glucose and water into gluconic acid and hydrogen peroxide (H2O2) using atmospheric oxygen. For many years glucose oxidases have been used in the baking industry as processing aids in particular to strengthen the gluten network. Active oxygen in the hydrogen peroxide oxidizes thiol groups (SH-groups) in the gluten protein into disulfide bridges (cysteine), thus strengthening the dough made from the flour. Active oxygen in the hydrogen peroxide oxidizes the thiol groups (SH-groups) present in cysteine in the gluten protein into disulfide bridges (cystine), thus strengthening the dough made from the flour.

Flour having low protein content is usually classified as weak. The gluten of weak flour is very extensible under stress but does not return to its original dimensions when the stress is removed. Flour with high protein content is classified as strong. The gluten of strong flour is less extensible than that of weak flour. It is more resistant to mixing.

Strong flour is often preferred for baking purposes, since the rheological and handling properties of dough prepared from such flour are superior to those obtained with weak flour. In addition, the shape and texture of a bakery product prepared from strong flour are remarkably better as compared with weak flour. A dough prepared from strong flour is also more stable as compared with that prepared from weak flour. This is one of the most important—if not the most important—properties in view of the baking process. A dough can be strengthened by using azodicarbonamide and the volume of a baked product from such a dough can be improved by adding bromate to the dough. However, these compounds are chemical oxidizers and their acceptance in food is decreasing. For example ADA is not authorised as a food additive in food in Europe and Australia. Potassium bromate is not authorised for use in food products in Europe, Canada and other countries. Therefore, alternatives for these oxidizers are desired.

### SUMMARY OF THE INVENTION

The present invention relates to the use of a Penicillium glucose oxidase as a chemical oxidizer replacement in the food industry.

In one aspect, the invention relates to the use of a composition comprising a Penicillium glucose oxidase as a chemical oxidizer replacement in the baking industry.

In another aspect, the invention relates to the use of a composition comprising a Penicillium glucose oxidase as a chemical oxidizer replacement in the baking industry.

In another aspect, the invention relates to the use of a Penicillium glucose oxidase to completely or partially replace bromate. In an aspect, the invention relates to the use of a Penicillium glucose oxidase to completely or partially replace potassium bromate. In an aspect, the invention relates to the use of a Penicillium glucose oxidase to completely or partially replace potassium bromate in flour, in a composition such as a baking composition, in dough, in a baking process or in the baking industry. In another aspect, the invention relates to the use of a Penicillium glucose oxidase to completely or partially replace azodicarbonamide (ADA). In another aspect, the invention relates to the use of a Penicillium glucose oxidase to completely or partially replace azodicarbonamide (ADA) in a baking process or in the baking industry. In another aspect, the invention relates to the use of a Penicillium glucose oxidase to completely or partially replace azodicarbonamide (ADA) in flour, in a composition such as baking composition, or in dough,

In another aspect, the invention relates to a pre-mix comprising Penicillium glucose oxidase and no or low levels of azodicarbonamide.

In an aspect, the invention relates to a pre-mix, such as a baking pre-mix, comprising Penicillium glucose oxidase and no or low levels of potassium bromate.

In another aspect, the invention relates a pre-mix comprising Penicillium glucose oxidase and no or low levels of bromate.

In another aspect, the invention relates to a method for improving flour, which method comprises adding to the flour a Penicillium glucose oxidase.

In another aspect, the invention relates to flour comprising a Penicillium glucose oxidase and no or low levels of azodicarbonamide or bromate. In another aspect, the invention relates to a flour comprising a Penicillium glucose oxidase and no or low levels of azodicarbonamide and/or potassium bromate.

In another aspect, the invention relates to a method for preparing a dough comprising combining a pre-mix according to the invention or a flour according the invention with at least one dough ingredient. In another aspect, the invention relates to a method to replace a chemical oxidiser in a baking product comprising the step of adding a Penicillium glucose oxidase, preferably as set out in amino acids 19-604 of SEQ ID No.1, to at least one dough ingredient.

In another aspect, the invention relates to a dough prepared from a pre-mix according to the invention or from a flour according to the invention.

In another aspect, the invention relates a method for preparing a baked product comprising the step of baking the dough according to the invention.

In another aspect, the invention relates to a baked product prepared from a pre-mix according to the invention, from a flour according to the invention or from a dough according to the invention.

In another aspect, the invention relates to a baked product prepared from a Penicillium glucose oxidase as described herein and one or more dough ingredients.

### DESCRIPTION OF THE SEQUENCE LISTING

SEQ ID No.1 sets out the amino acid sequence of the Penicillium glucose oxidase used in the method according to the invention. Amino acids 1 to 18 represent the signal sequence used for secretion of the Penicillium glucose oxidase enzyme (amino acids 19-604).

SEQ ID No.2 sets out the cDNA sequence encoding the Penicillium glucose oxidase depicted in FIG. 1. Nucleotides 1 to 54 encode the signal sequence, nucleotides 55 to 1816 encode the mature glucose oxidase, including a translational termination sequence (5′-TAAA-3′) at the 3′-terminus.

### SHORT DESCRIPTION OF THE FIGURES

FIG. 1. Amino acid sequence of the Penicillium glucose oxidase used in the method according to the invention. Amino acids 1 to 18 represent the signal sequence used for secretion of the glucose oxidase (amino acids 19-604).

FIG. 2. cDNA sequence encoding the Penicillium glucose oxidase depicted in FIG. 1. Nucleotides 1 to 54 encode the signal sequence, nucleotides 55 to 1816 encode the mature glucose oxidase, including a translational termination sequence (5′-TAAA-3′) at the 3′-terminus.

FIG. 3 C-cell structural analysis of shape and crumb structure of bread in which azodicarbonamide (ADA) or Penicillium glucose oxidase is used in the invention. 1A: control; 1B: 40 ppm ADA; 2A: 10 ppm Penicillium glucose oxidase; 2B: 40 ppm Penicillium glucose oxidase; 3A: 40 ppm ADA; 3B: 20 ppm Penicillium glucose oxidase.

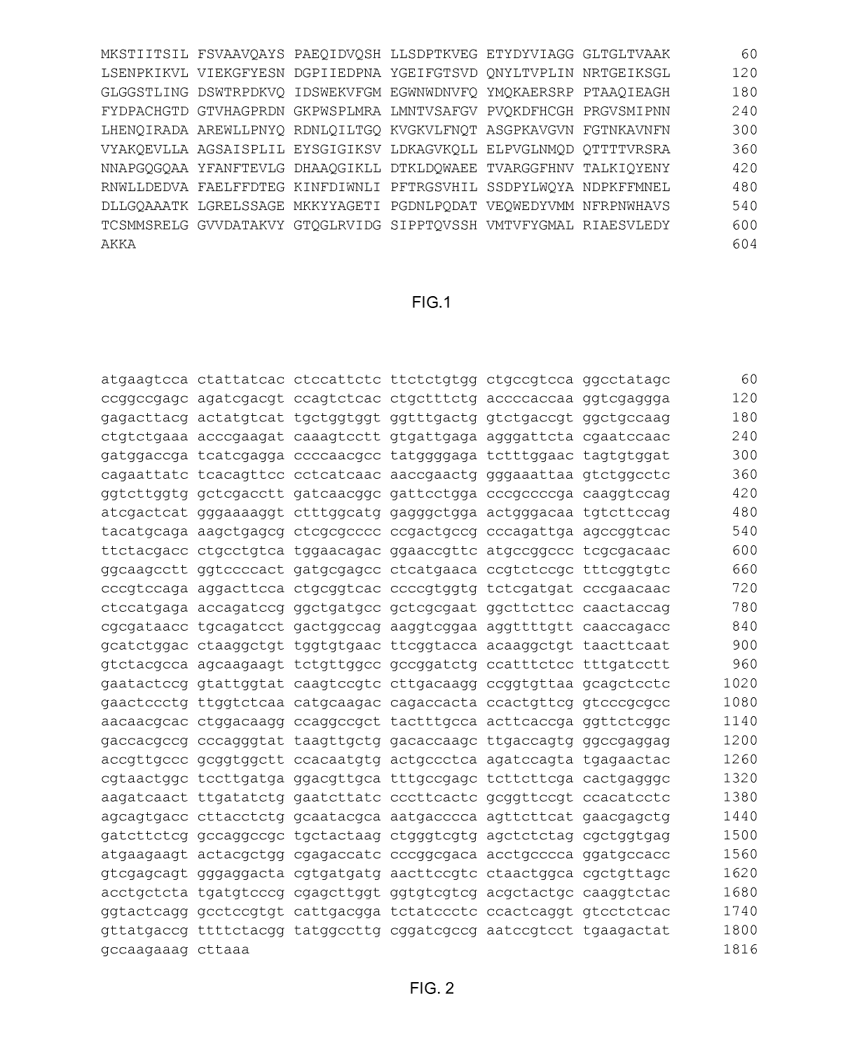

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The present invention relates to the use of a Penicillium glucose oxidase as a chemical oxidizer replacement in the baking industry. The present invention also relates to the use a composition comprising a Penicillium oxidase as a chemical oxidizer replacement in the baking industry. The use according to the invention has the advantage that it obviates the use of chemical oxidizers, such as bromate and azodicarbonamide, which are included in flour or dough to strengthen the dough and increase the volume of a baked product produced from the dough.

In one embodiment of the invention, the Penicillium glucose oxidase completely or partially replaces bromate. In the context of the present invention, ‘bromate’ refers to bromate salts typically used in the baking industry, such as potassium bromate. In one embodiment, the glucose oxidase replaces at least 10%, at least 20%, at least 30%, at least 40%, at least 50%, at least 60%, at least 70%, at least 80%, at least 90% of the bromate otherwise used in the baking process. In another embodiment, the glucose oxidase replaces 100% of the bromate otherwise used in the baking process. Replacement of bromate by a Penicillium glucose oxidase may be done without deterioration of process tolerance, dough handling, crumb quality, loaf volume and product quality of the baked product.

In another embodiment of the invention, the Penicillium glucose oxidase completely or partially replaces azodicarbonamide (ADA). In one embodiment, the Penicillium glucose oxidase replaces at least 10%, at least 20%, at least 30%, at least 40%, at least 50%, at least 60%, at least 70%, at least 80%, at least 90% of the ADA otherwise used in the baking process. In another embodiment, the Penicillium glucose oxidase replaces 100% of the ADA otherwise used in the baking process.

In one embodiment of the invention, the Penicillium glucose oxidase completely or partially replaces bromate. In the context of the present invention, ‘bromate’ refers to bromate salts typically used in the baking industry, such as potassium bromate. In one embodiment, the glucose oxidase replaces by weight at least 10%, at least 20%, at least 30%, at least 40%, at least 50%, at least 60%, at least 70%, at least 80%, at least 90% of the bromate otherwise used in the baking process. In another embodiment, the glucose oxidase replaces 100% by weight of the bromate otherwise used in the baking process. Replacement of bromate by a Penicillium glucose oxidase may be done without deterioration of process tolerance, dough handling, crumb quality, loaf volume and product quality of the baked product.

In another embodiment of the invention, the Penicillium glucose oxidase completely or partially replaces azodicarbonamide (ADA). In one embodiment, the Penicillium glucose oxidase replaces by weight at least 10%, at least 20%, at least 30%, at least 40%, at least 50%, at least 60%, at least 70%, at least 80%, at least 90% of the ADA otherwise used in the baking process. In another embodiment, the Penicillium glucose oxidase replaces 100% by weight of the ADA otherwise used in the baking process.

A Penicillium glucose oxidase for use according to the invention may be obtained by any suitable means. In one embodiment, it is isolated from a source, in particular a prokaryotic or eukaryotic microorganism, containing the enzyme. Suitable examples of microorganisms are mammalian, plant, fungal and algal microorganisms as mentioned below. In one embodiment, the Penicillium glucose is isolated from a Penicillium species, in particular from Penicillium chrysogenum. In one embodiment, the Penicillium glucose oxidase is isolated from a Penicillium species, in particular from Penicillium chrysogenum. A suitable example of such a Penicillium glucose oxidase is a Penicillium glucose oxidase having a sequence as depicted in SEQ ID No. 1. or a glucose oxidase which has an amino acid sequence which shows at least 75% identity to amino acids 19-604 of SEQ ID No.1. In one embodiment, the glucose oxidase has an amino acid sequence which shows at least 80%, at least 85%, at least 90%, at least 95%, at least 97%, at least 98% or at least 99% identity to amino acids 19-604 of SEQ ID No.1. In another embodiment, the Penicillium glucose oxidase is generated using standard molecular biology techniques, e.g. by de novo synthesis of a nucleotide sequence which encodes a glucose oxidase which comprises an amino acid sequence which shows at least 75% identity, at least 80%, at least 85%, at least 90%, at least 95%, at least 97%, at least 98% or at least 99% identity to amino acids 19-604 of SEQ ID No.1. In one embodiment, a nucleotide sequence according to SEQ ID No. 2 is used.

Such a nucleotide sequence may then be used for transformation of a host microorganism. In another embodiment, the Penicillium glucose oxidase is generated using site-saturation mutagenesis, scanning mutagenesis, insertional mutagenesis, random mutagenesis, site-directed mutagenesis, and directed-evolution of an existing glucose oxidase, e.g. of the one depicted in SEQ ID No.1, as well as various other recombination approaches known to a skilled person in the art. Glucose oxidases (EC 1.1.3.4) catalyse the oxidation of glucose into gluconic acid and hydrogen peroxide using atmospheric oxygen.

In the context of the present invention, in order to determine the percentage of sequence identity of two amino acid sequences or of two nucleic acid sequences, the sequences are aligned for optimal comparison purposes. In order to optimize the alignment between the two sequences, gaps may be introduced in any of the two sequences that are compared. Such alignment may be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. Upon the alignment of two sequences, the resulting aligned amino acid positions are usually referred to as corresponding positions. The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) p. 276-277, http://emboss.bioinformatics.nl/). For protein sequences EBLOSUM62 is used for the substitution matrix. For nucleotide sequences, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences similar to nucleic acid molecules of the invention. BLAST protein searches can be performed with the XBLAST program, score=50, word length=3 to obtain amino acid sequences similar to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

Any microorganism may be used as a recombinant host cell to produce a Penicillium glucose oxidase to be used in the invention. In one embodiment, the microorganism is a prokaryotic cell, such as a Gram-negative or Gram-positive bacterium. Suitable bacteria include Escherichia, Anabaena, Caulobactert, Cyanobacteria, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Moraxella, Neisseria, Staphylococcus, Streptomyces or Thermoactinomyces. In another embodiment, the microorganism is a eukaryotic cell, such as a mammalian cell, insect cell, plant cell, fungal cell or algal cell. Suitable examples of mammalian cells are CHO cells, COS cells, 293 cells, Per.C6® cells, and hybridomas. Suitable examples of insect cells include Sf9 and Sf21 cells and derivatives thereof. Suitable examples of fungal cells include yeast cells, such as Candida, Hansenula, Kluyveromyces, Pichia, Saccharomyces, Schizosaccharomyces, or Yarrowia strain; more preferably Kluyveromyces lactis, S. cerevisiae, Hansenula polymorpha, Yarrowia lipolytica and Pichia pastoris, or a filamentous fungi. Filamentous fungi include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). Filamentous fungal strains include strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Rasamsonia, Schizophyllum, Talaromyces, Thermoascus, Thielavia, Tolypocladium, and Trichoderma. Preferred filamentous fungal cells belong to a species of an Aspergillus, Chrysosporium, Myceliophthora, Penicillium, Talaromyces, Fusarium, Rasamsonia, Thermoascus or Trichoderma genus, and most preferably a species of Aspergillus niger, Aspergillus awamori, Aspergillus foetidus, Aspergillus sojae, Aspergillus fumigatus, Aspergillus oryzae, Aspergillus tubingensis, Chrysosporium lucknowense, Fusarium oxysporum, Myceliophthora thermophila, Rasamsonia emersonii, Trichoderma reesei, Talaromyces emersonii, Thermoascus aurantiacus or Penicillium chrysogenum. Algae is the group of unicellular and multicellular eukaryortic photosynthetic organisms, including microalgae, such as Dunaliella, Spirulina and Chlorella. Algae include algal host cells and microalgae, such as Schizochitrium. In one embodiment, the recombinant host cell comprising a nucleotide sequence or a nucleic acid molecule according to the invention is an Aspergillus, Bacillus, Chrysosporium, Escherichia, Kluyveromyces, Myceliophthora, Penicillium, Pseudomonas, Rasamsonia, Saccharomyces, Streptomyces or Talaromyces species, preferably a Bacillus subtilis, Bacillus amyloliquefaciens, Bacillus licheniformis, Escherichia coli, Aspergillus niger, Aspergillus oryzae, Myceliophthora thermophila, Rasamsonia emersonii or Trichoderma reesei species. The recombinant host cell is preferably capable of expressing or overexpressing the glucose oxidase, for example as described in WO 2010/121933 or WO 2012/001169. The recombinant host cell, such as a host cell belonging to the genus Aspergillus, may further comprise one or more modifications in its genome such that the recombinant microbial host cell is deficient in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, agsE or amyC if compared to a parent host cell and measured under the same conditions. Suitable methods of producing said host cells include the ones described in WO2012/001169 and WO20140/13074.

The glucose oxidase used in the present invention may be a fusion protein. Techniques for producing fusion polypeptides are known in the art, and include ligating the coding sequences encoding the polypeptides so that they are in frame. Expression of the fused polypeptide is under control of the same promoter (s) and terminator. The hybrid polypeptides may comprise a combination of partial or complete polypeptide sequences obtained from at least two different polypeptides wherein one or more may be heterologous to a host cell. Such fusion polypeptides from at least two different polypeptides may comprise a binding domain from one polypeptide, such as a starch binding domain or a carbohydrate binding domain, operably linked to a catalytic domain from a second polypeptide. Examples of fusion polypeptides and signal sequence fusions are for example as described in WO2010/121933, WO2013/007820 and WO2013/007821.

In the context of the present invention the term ‘polypeptide’ refers to a molecule which contains a backbone of a chain of at least ten amino acids, wherein the amino acids are covalently linked to each other by peptide bonds. These backbone amino acids groups may be linked to other groups, such as other amino acid sequences, sugar groups or lipid groups. The polypeptide may contain structural features, such as alpha-helices, beta-pleated sheets or disulphide bridges. A polypeptide used according to the present invention may comprise a catalytic domain and one or more binding domains, such as a starch or carbohydrate binding domain. In the context of the present invention, the amino acid sequence is also referred to as ‘polypeptide sequence’ or ‘protein sequence’. The term ‘polypeptide’ includes proteins.

In the Figures and in other places of the description of the present invention, the one letter code for amino acids is used, where A stands for Alanine, I for Isoleucine, L for Leucine, V for Valine, S for Serine, G for Glycine, P for Proline, Q for Glutamine, E for Glutamic acid, R for Arginine, D for Aspartic acid, K for Lysine, N for Asparagine, Y for Tyrosine, H for Histidine, F for Phenylalanine, C for Cysteine, T for Threonine. In the one letter code for amino acids M stands for Methionine, W for Tryptophan. Such one letter codes are commonly known in the art, see e.g. Sambrook, et al. (Molecular Cloning: A Laboratory Manual, 2^{nd} ed. Cold Spring Harbor Laboratory, Cold Spring Harbor Laboratory Press, Cold Spring Harbor, N.Y., 1989).

The Penicillium glucose oxidase for use according to the invention may be comprised in a composition.

According to another aspect, the invention relates to a composition comprising a glucose oxidase having an amino acid sequence having at least 75% identity to amino acids 19-604 of SEQ ID No. 1 and a dough ingredient and an additional enzyme.

According to another aspect, the invention relates to a composition comprising a glucose oxidase having an amino acid sequence having at least 75% identity to amino acids 19-604 of SEQ ID No. 1 and a dough ingredient.

According to another aspect, the invention relates to a composition comprising a glucose oxidase having an amino acid sequence having at least 75% identity to amino acids 19-604 of SEQ ID No. 1 and an additional enzyme.

The composition may comprise between 0.001% and 100% w/w of a Penicillium glucose oxidase based on total protein. Preferably, the composition comprises between 1% and 70% w/w of the glucose oxidase based on total protein. In one embodiment, the preparation comprises between 1% and 50% w/w of the glucose oxidase based on total protein. In yet another embodiment, the preparation comprises between 1% and 30% w/w of a Penicillium glucose oxidase based on total protein. In yet another embodiment, the preparation comprises between 5% and 20% w/w of a Penicillium glucose oxidase based on total protein.

The composition may comprise between 0.001% and 100% w/w of a Penicillium glucose oxidase based on total enzyme protein. Total enzyme protein refers to all enzyme protein present in the composition. Preferably, the composition comprises between 1% and 70% w/w of the glucose oxidase based on total enzyme protein. In one embodiment, the preparation comprises between 1% and 50% w/w of the glucose oxidase based on total enzyme protein. In yet another embodiment, the preparation comprises between 1% and 30% w/w of a Penicillium glucose oxidase based on total enzyme protein. In yet another embodiment, the preparation comprises between 5% and 20% w/w of a Penicillium glucose oxidase based on total enzyme protein.

In one embodiment of the invention, the Penicillium glucose oxidase is the only enzymatic component in the preparation. In one embodiment of the invention, the Penicillium glucose oxidase is the only enzymatic component in the composition. In another embodiment of the invention, the composition comprises a Penicillium glucose oxidase and at least one additional enzymatic activities. The at least one additional enzyme activity may be selected from enzymes like a protease, such as a endoprotease or an exoprotease; a peptidase, such as an exopeptidase or an endopeptidase; a lipolytic enzyme, such as a triacyl glycerol lipase, a phospholipase, a galactolipase or an enzyme having both phospholipase and galactolipase activity; or a carbohydrase, such as a cellulase, a hemicellulase, in particular a pentosanase such as a xylanase; a cross-linking enzyme, such as a transglutaminase; a maltogenic alpha amylase; an alpha amylase; a beta amylase; an amyloglucosidase, an oxidase, a peroxidase, a hexose oxidase, a sulfhydryl oxidase, a pyranose oxidase, a laccase; a protein disulfide isomerase; an asparaginase.

In one embodiment, the flour improving composition further comprises one or more components selected from the group consisting of milk powder, gluten, granulated fat, an additional enzyme, an amino acid, a salt, an oxidant such as ascorbic acid, a reducing agent such as L-cysteine, an emulsifier such as mono-glycerides, di-glycerides, glycerol monostearate, sodium stearoyl lactylate, calcium stearoyl lactylate, polyglycerol esters of fatty acids and diacetyl tartaric acid esters of mono- and diglycerides, gums such as guar gum and xanthan gum, flavours, acids such as citric acid and propionic acid, starch, modified starch, gluten, humectants such as glycerol, and preservatives.

The flour improving composition may comprise propylene glycerol monostearate,

In one embodiment, the composition according to the invention is a flour improving composition which further comprises one or more components selected from the group consisting of milk powder, gluten, granulated fat, an additional enzyme, an amino acid, a salt, an oxidant such as ascorbic acid, a reducing agent such as L-cysteine, an emulsifier such as mono-glycerides, di-glycerides, glycerol monostearate, sodium stearoyl lactylate, calcium stearoyl lactylate, polyglycerol esters of fatty acids and diacetyl tartaric acid esters of mono- and diglycerides, gums such as guar gum and xanthan gum, flavours, acids such as citric acid and propionic acid, starch, modified starch, gluten, humectants such as glycerol, and preservatives. The flour improving composition may comprise propylene glycerol monostearate,

In one embodiment, the invention relates to a composition comprising the Penicillium glucose oxidase as described herein and one or more components selected from the group consisting of milk powder, gluten, granulated fat, an additional enzyme, an amino acid, a salt, an oxidant such as ascorbic acid, a reducing agent such as L-cysteine, an emulsifier such as mono-glycerides, di-glycerides, glycerol monostearate, sodium stearoyl lactylate, calcium stearoyl lactylate, polyglycerol esters of fatty acids and diacetyl tartaric acid esters of mono- and diglycerides, gums such as guargum and xanthangum, flavours, acids such as citric acid and propionic acid, starch, modified starch, gluten, humectants such as glycerol, and preservatives. The additional enzyme may be selected from enzymes like a protease, such as a endoprotease or an exoprotease; a peptidase, such as an exopeptidase or an endopeptidase; a lipolytic enzyme, such as a triacyl glycerol lipase, a phospholipase, a galactolipase or an enzyme having both phospholipase and galactolipase activity; or a carbohydrase, such as a cellulase, a hemicellulase, in particular a pentosanase such as a xylanase; a cross-linking enzyme, such as a transglutaminase; a maltogenic alpha amylase; a further amylase such as a further alpha amylase or a beta amylase; an oxidase, a peroxidase, an hexose oxidase, a glucose oxidase, a laccase; a protein disulfide isomerase; an asparaginase

In another embodiment, the composition comprises a lipolytic enzyme, preferably a phospholipase. In another aspect, the composition comprises the Penicillium glucose oxidase as described herein and a lipolytic enzyme, preferably a phospholipase.

In another aspect, the composition comprises the Penicillium glucose oxidase as described herein and ascorbic acid.

In another aspect, the composition comprises the Penicillium glucose oxidase as described herein and a amyloglucosidase.

In another aspect, the composition comprises the Penicillium glucose oxidase as described herein and a hemicellulase.

In another aspect, the composition comprises the Penicillium glucose oxidase as described herein and a fungal alpha amylase.

In another embodiment, the composition comprises a Penicillium glucose oxidase with an amino acid sequence having at least 75%, at least 80%, at least 85%, at least 90%, at least 95%, at least 97%, at least 98% or at least 99% identity to amino acids 19-604 of SEQ ID No.1 and ascorbic acid.

In another embodiment, the composition comprises a Penicillium glucose oxidase with an amino acid sequence having at least 75%, at least 80%, at least 85%, at least 90%, at least 95%, at least 97%, at least 98% or at least 99% identity to amino acids 19-604 of SEQ ID No.1, ascorbic acid and an amyloglucosidase.

In yet another embodiment, the composition comprises a Penicillium glucose oxidase with an amino acid sequence having at least 75%, at least 80%, at least 85%, at least 90%, at least 95%, at least 97%, at least 98% or at least 99% identity to amino acids 19-604 of SEQ ID No.1, a bacterial or fungal hemicellulase and a bacterial or fungal alpha amylase.

The composition according to the invention may be formulated in any suitable form. In one embodiment, the composition according to the invention is formulated in a dry form, such as a freeze-dried or spray-dried powder or a granulate. In another embodiment, the composition according to the invention is in liquid form, such as in the form of an emulsion, a suspension or a solution. In yet another embodiment, the composition according to the invention is a paste or a gel.

The Penicillium glucose oxidase may be included in a flour or pre-mix to obtain a flour or pre-mix with no or low levels of azodicarbonamide or no or low levels of bromate.

In an aspect, the invention relates to a pre-mix, such as a baking pre-mix, comprising Penicillium glucose oxidase and no or low levels of potassium bromate. In another aspect, the invention relates to flour comprising a Penicillium glucose oxidase and no or low levels of azodicarbonamide and/or potassium bromate.

In an aspect the invention relates to a method to replace a chemical oxidiser in a baking product comprising the step of adding Penicillium glucose oxidase, preferably as set out in amino acids 19-604 of SEQ ID No.1, as an ingredient in the preparation of said product.

In an aspect the invention relates to a method to replace a chemical oxidiser in a baking product comprising the step of adding a Penicillium glucose oxidase, preferably as set out in amino acids 19-604 of SEQ ID No.1, to at least one dough ingredient. In an aspect of the method according to the invention the at least one dough ingredient is ascorbic acid. In an aspect of the invention the at least one dough ingredient is at least one of amyloglucosidase, bacterial or fungal hemicellulase, and bacterial or fungal alpha amylase.

In aspect of the invention the Penicillium glucose oxidase is part of a composition further comprising one or more components selected from the group consisting of milk powder, gluten, granulated fat, an additional enzyme, an amino acid, a salt, an oxidant such as ascorbic acid, a reducing agent such as L-cysteine, an emulsifier such as lecithine, mono-glycerides, di-glycerides, glycerol monostearate, sodium stearoyl lactylate, calcium stearoyl lactylate, polyglycerol esters of fatty acids and diacetyl tartaric acid esters of mono- and diglycerides, gums such as guar gum and xanthan gum, flavours, acids such as citric acid and propionic acid, starch, modified starch, gluten, humectants such as glycerol, and preservatives.

In an aspect the invention relates to a method to replace a chemical oxidiser in a baking product comprising the step of adding a composition comprising Penicillium glucose oxidase, preferably as set out in amino acids 19-604 of SEQ ID No.1, as an ingredient in the preparation of said product.

In an aspect the invention relates to a method to replace a chemical oxidiser in a baking product comprising the step of adding a composition comprising Penicillium glucose oxidase, preferably as set out in amino acids 19-604 of SEQ ID No.1, to at least one dough ingredient. In an aspect of the invention the at least one dough ingredient is ascorbic acid.

In an aspect of the invention, the composition further comprises ascorbic acid. In an aspect of the invention, the composition further comprises at least one of amyloglucosidase, bacterial or fungal hemicellulase, and bacterial or fungal alpha amylase.

In an aspect of the invention, the method of the invention is a method to completely or partially replace bromate.

In an aspect of the invention, the method of the invention is a method to completely or partially replace azodicarbonamide (ADA).

In another aspect, the invention relates to a dough comprising a Penicillium glucose oxidase, said glucose oxidase comprising an amino acid sequence having at least 75% identity to amino acids 19-604 of SEQ ID No. 1 and one or more dough ingredients. In another aspect, the invention relates a method for preparing a baked product comprising the step of baking the dough according to the invention.

In another aspect, the invention relates to a baked product prepared from a Penicillium glucose oxidase and one or more dough ingredients, from a pre-mix according to the invention, from a flour according to the invention or from a dough according to the invention.

Therefore, in another aspect, the present invention relates to a flour comprising by weight of flour:

(i) 1-50 ppm of a glucose oxidase with a protein sequence having at least 75%, at least 80%, at least 85%, at least 90%, at least 95%, at least 97%, at least 98% or at least 99% identity to amino acids 19-604 of SEQ ID No.1
(ii) 0 to 20 ppm azodicarbonamide and
(iii) 0 to 30 ppm bromate.

In another embodiment, the present invention relates to a flour comprising by weight of flour
(i) 1-50 ppm of a glucose oxidase with a protein sequence having at least 75%, at least 80%, at least 85%, at least 90%, at least 95%, at least 97%, at least 98% or at least 99% identity to amino acids 19-604 of SEQ ID No.1
(ii) 10-50 ppm ascorbic acid
(iii) 0-20 ppm amyloglucosidase
(iv) 0 to 20 ppm azodicarbonamide and
(v) 0 to 30 ppm bromate.

In another embodiment, the present invention relates to a flour comprising by weight of flour
(i) 1-50 ppm of a glucose oxidase with a protein sequence having at least 75%, at least 80%, at least 85%, at least 90%, at least 95%, at least 97%, at least 98% or at least 99% identity to amino acids 19-604 of SEQ ID No.1
(ii) 10-50 ppm ascorbic acid
(iii) 1-20 ppm fungal alpha amylase
(iv) 1-50 ppm hemicellulase
(v) 0 to 20 ppm azodicarbonamide and
(vi) 0 to 30 ppm bromate.

In another aspect, the present invention relates to a flour comprising by weight of flour:

(i) 1-50 ppm of a Penicillium glucose oxidase comprising an amino acid sequence having at least 75%, at least 80%, at least 85%, at least 90%, at least 95%, at least 97%, at least 98% or at least 99% identity to amino acids 19-604 of SEQ ID No.1
(ii) 0 to 20 ppm azodicarbonamide and
(iii) 0 to 30 ppm bromate.

In another embodiment, the present invention relates to a flour comprising by weight of flour
(i) 1-50 ppm of a Penicillium glucose oxidase comprising an amino acid sequence having at least 75%, at least 80%, at least 85%, at least 90%, at least 95%, at least 97%, at least 98% or at least 99% identity to amino acids 19-604 of SEQ ID No.1
(ii) 10-50 ppm ascorbic acid
(iii) 0-20 ppm amyloglucosidase
(iv) 0 to 20 ppm azodicarbonamide and
(v) 0 to 30 ppm bromate.

In another embodiment, the present invention relates to a flour comprising by weight of flour
(i) 1-50 ppm of a Penicillium glucose oxidase comprising an amino acid sequence having at least 75%, at least 80%, at least 85%, at least 90%, at least 95%, at least 97%, at least 98% or at least 99% identity to amino acids 19-604 of SEQ ID No.1
(ii) 10-50 ppm ascorbic acid
(iii) 1-20 ppm fungal alpha amylase
(iv) 1-50 ppm hemicellulase
(v) 0 to 20 ppm azodicarbonamide and
(vi) 0 to 30 ppm bromate.

The Penicillium glucose oxidase typically has an activity in the range of about 2500 to 4000 glucose oxidase units/gram enzyme when determined according to the assay described below. In one embodiment, the Penicillium glucose oxidase has an activity in the range of about 2800 to 4000 glucose oxidase units/gram enzyme. In another embodiment, the Penicillium glucose oxidase has an activity in the range of about 3000 to 4000 glucose oxidase units/gram enzyme. In yet another embodiment, the Penicillium glucose oxidase has an activity in the range of about 3000 to 3600 glucose oxidase units/gram enzyme. In yet another embodiment, the Penicillium glucose oxidase has an activity of about 3300 glucose oxidase units/gram enzyme. Glucose oxidase activity is determined in an assay in which gluconic acid formed is titrated. 1 ml of diluted glucose oxidase is added to 25 ml of preheated 30 g/I glucose monohydrate solution at 35 degrees C. Sample dilutions and substrate are prepared in 50 mM HAc buffer at pH 5.1, containing 50 mM NaCl. After 15 minutes incubation at 35 degrees C., the reaction is terminated by the addition of 10 ml 0.1 N NaOH, at the same time neutralizing the gluconic acid formed. Excess NaOH is titrated with 0.05 M HCl. The difference in HCl consumption between a sample and blank run is a measure for the amount of glucose oxidase activity. One glucose oxidase unit is defined as the amount of enzyme needed to oxidize 3 mg of glucose to gluconic acid under conditions of the assay.

Glucose oxidase activities may be determined using alternative assays, such as spectrophotometrically at 450 nm using o-dianisidine as described by Witteveen et al. 1990 (“Glucose oxidase overproducing and negative mutants of Aspergillus niger”, Appl. Microbiol. Biotechnol 33:683-686) or a glucose oxidase assay as described in the Food Chemicals Codex (FCC).

The term “pre-mix” is defined herein to be understood in its conventional meaning, i.e. as a mix of baking agents, generally including flour, which may be used not only in industrial bread-baking plants/facilities, but also in retail bakeries. The pre-mix may be prepared by mixing the glucose oxidase or a composition according to the invention with a suitable carrier such as flour, starch or a salt. The pre-mix may contain additives as mentioned herein.

In another aspect, the present invention relates to a method for improving flour, wherein the method comprises adding to the flour (i) 1 to 50 ppm of a Penicillium glucose oxidase, for example one having an amino acid composition which shows at least 75% identity to amino acids 19-604 of SEQ ID No.1, or (ii) a composition according to the invention. Improvements are always relative to flour without a Penicillium glucose oxidase. In one embodiment 5-45 ppm by weight of flour is used, in another embodiment 10-40 ppm by weight of flour is used. In one embodiment, 1 ppm-50 ppm by weight of flour of a Penicillium glucose oxidase having an activity in a range of about 2500-4000, of about 2800-4000, of about 3000-4000, or of about 3000-3600 glucose oxidase units/gram enzyme, is used to improve the flour. In one embodiment, the flour is a weak flour, i.e. a flour which is low in protein content or showing lack of dough stability in the bread making process.

In another aspect, the present invention relates to a method for improving flour, wherein the method comprises adding to the flour (i) 1-50 ppm of a Penicillium glucose oxidase comprising an amino acid sequence having at least 75%, at least 80%, at least 85%, at least 90%, at least 95%, at least 97%, at least 98% or at least 99% identity to amino acids 19-604 of SEQ ID No.1 or (ii) a composition according to the invention. Improvements are always relative to flour without the Penicillium glucose oxidase. In one embodiment 5-45 ppm of the Penicillium glucose oxidase by weight of flour is used, In another embodiment 10-40 ppm by weight of flour is used. In one embodiment, 1 ppm-50 ppm by weight of flour of a Penicillium glucose oxidase having an activity in a range of about 2500-4000, of about 2800-4000, of about 3000-4000, or of about 3000-3600 glucose oxidase units/gram enzyme, is used to improve the flour.

A flour or pre-mix according to the invention may be used to produce a dough according to the invention. Therefore, in yet another aspect, the present invention relates to a dough prepared from a flour or a pre-mix comprising a Penicillium glucose oxidase, for example one having an amino acid composition which shows at least 75% identity to amino acids 19-604 of SEQ ID No.1, and to a dough comprising a Penicillium glucose oxidase, for example one having an amino acid composition which shows at least 75% identity to amino acids 19-604 of SEQ ID No.1, and no or low levels of azodicarbonamide or no or low levels of bromate. In one embodiment, the dough is prepared from flour comprising 1 to 50 ppm of a Penicillium glucose oxidase and 0 to 20 ppm azodicarbonamide and 0 to 30 ppm bromate.

In one embodiment, the dough is prepared from flour comprising 1 to 50 ppm based on flour weight of a Penicillium glucose oxidase comprising an amino acid sequence having at least 75% identity to amino acids 19-604 of SEQ ID No. 1 and 0 to 20 ppm azodicarbonamide based on flour weight and 0 to 30 ppm potassium bromate based on flour weight. In another embodiment, the dough is prepared from flour comprising 1 to 50 ppm of a Penicillium glucose oxidase and 0 ppm azodicarbonamide and 0 ppm bromate. In another embodiment, the dough is prepared from flour comprising 1 to 50 ppm based on flour weight of a Penicillium glucose oxidase comprising an amino acid sequence having at least 75% identity to amino acids 19-604 of SEQ ID No. 1, and 0 ppm azodicarbonamide based on flour weight and 0 ppm potassium bromate based on flour weight.

In the context of the present invention, the term ‘dough’ is defined as a mixture of flour and other ingredients. In the context of the present invention, the term ‘dough’ is defined as a mixture of flour and dough ingredients. In one embodiment, the dough is firm enough to knead or roll. The dough may be fresh prepared or par-baked. Dough is made using at least one dough ingredient. The at least one dough ingredient may be selected from the group consisting of (cereal) flour, a lecithin source including egg, water, salt, sugar, flavours, a fat source including butter, margarine, oil and shortening, baker's yeast, chemical leavening systems such as a combination of an acid (generating compound) and bicarbonate, a protein source including milk, soy flour, non-chemical oxidants (including ascorbic acid), a reducing agent (including L-cysteine), an emulsifier (including mono- and di glycerides, monoglycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM), propylene glycerol monostearate and lecithin), a gum (including guar gum and xanthan gum), a flavour, an acids (including citric acid, propionic acid), a starch, a modified starch, gluten, a humectant (including glycerol) and a preservative.

Dough is made using dough ingredients, which include without limitation (cereal) flour, a lecithin source including egg, water, salt, sugar, flavours, a fat source including butter, margarine, oil and shortening, baker's yeast, chemical leavening systems such as a combination of an acid (generating compound) and bicarbonate, a protein source including milk, soy flour, non-chemical oxidants (including ascorbic acid), reducing agents (including L-cysteine), emulsifiers (including mono/di glycerides, monoglycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM), gums (including guar gum and xanthan gum), flavours, acids (including citric acid, propionic acid), starch, modified starch, gluten, humectants (including glycerol) and preservatives. The use of chemical oxidizers such as bromate and azodicarbonamide (ADA) is optional and not necessary anymore if a Penicillium glucose oxidase is included. In one embodiment, a Penicillium glucose oxidase with a protein sequence having at least 75%, at least 80%, at least 85%, at least 90%, at least 95%, at least 97%, at least 98% or at least 99% identity to the sequence as depicted in SEQ ID No.1 is included. For leavened products, primarily baker's yeast is used next to chemical leavening systems such as a combination of an acid or acid generating compound and bicarbonate.

Dough is usually made from basic dough ingredients including (cereal) flour, such as wheat flour or rice flour, water and optionally salt. Cereals include maize, rice, wheat, barley, sorghum, millet, oats, rye, triticale, buckwheat, quinoa, spelt, einkorn, emmer, durum and kamut.

In the context of the present invention, the term dough includes a batter. A batter is a semi-liquid mixture, being thin enough to drop or pour from a spoon, of one or more flours combined with liquids such as water, milk or eggs used to prepare various foods, including cake. A batter is typically made using flour combined with a liquid source such as water, milk or eggs used to prepare a cake. The dough may be made using a mix including a cake mix, a biscuit mix, a brownie mix, a bread mix, a pancake mix and a crepe mix.

In one embodiment, a composition according to the invention is added to a dough. The composition according to the invention may be provided in a dry form, to allow easy addition to the dough, but liquid forms are also possible. A liquid form includes without limitation an emulsion, a suspension and a solution. Irrespective of the formulation of the enzyme composition, any additive or additives known to be useful in the art to improve and/or maintain the enzyme's activity, the quality of the dough and/or the baked product may be applied.

Yeast, enzymes and optionally additives are generally added separately from each other to the dough. Enzymes may be added in a dry, e.g. granulated form, in a liquid form or in the form of a paste. Additives are in most cases added in powder form. Suitable additives include non-chemical oxidants, including ascorbic acid, reducing agents, including L-cysteine; emulsifiers, including lecithin and mono and diglycerides, such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM); gums, including guar gum and xanthan gum; flavours, acids, including citric acid and propionic acid; starch, including modified starch; gluten; humectants, including glycerol; and preservatives. A suitable additive includes propylene glycerol monostearate (PGMS).

The preparation of a dough from the dough ingredients is well known in the art and includes mixing of said ingredients and optionally one or more moulding and fermentation steps. In another aspect, the present invention relates to a method for improving a dough. The method comprises combining a pre-mix or flour according to the invention with at least one dough ingredient and is also encompassed by the present invention.

The preparation of baked products from such doughs is also well known in the art and may comprise moulding and shaping and further fermentation of the dough followed by baking at required temperatures and baking times. In one embodiment the invention provides a method to prepare a baked product comprising the step of baking a dough comprising a polypeptide according to the invention. The baking of the dough to produce a baked product may be performed using methods well known in the art. The invention also provides a baked product obtainable according to this method. In one embodiment of the invention, the baked product according to the invention is bread or cake.

Examples of baked products, whether of a white, brown or whole-meal type, which may be advantageously produced by the present invention include bread (in particular white, whole-meal or rye bread), typically in the form of loaves or rolls, French baguette-type bread, pastries, croissants, brioche, panettone, pasta, noodles (boiled or (stir-)fried), pita bread and other flat breads, tortillas, tacos, cakes, pancakes, cookies in particular biscuits, doughnuts, including yeasted doughnuts, bagels, pie crusts, steamed bread, crisp bread, brownies, sheet cakes, snack foods (e.g., pretzels, tortilla chips, fabricated snacks, fabricated potato crisps). The term baked product includes, bread containing from 2 to 30 wt % sugar, fruit containing bread, breakfast cereals, cereal bars, eggless cake, soft rolls and gluten-free bread. Gluten free bread herein and herein after is bread than contains at most 20 ppm gluten. Several grains and starch sources are considered acceptable for a gluten-free diet. Frequently used sources are potatoes, rice and tapioca (derived from cassava). Baked product includes without limitation tin bread, loaves of bread, twists, buns, such as hamburger buns or steamed buns, chapati, rusk, dried steam bun slice, bread crumb, matzos, focaccia, melba toast, zwieback, croutons, soft pretzels, soft and hard bread, bread sticks, yeast leavened and chemically-leavened bread, laminated dough products such as Danish pastry, croissants or puff pastry products, muffins, Danish bagels, confectionery coatings, crackers, wafers, pizza crusts, tortillas, pasta products, crepes, waffles, par-baked products and refrigerated dough products.

An example of a par-baked product includes, without limitation, partially baked bread that is completed at point of sale or consumption with a short second baking process.

The bread may be white or brown pan bread and may for example be manufactured using a so called American style Sponge and Dough method or an American style Direct method.

The American Style sponge and dough method as referred to herein is a two-step bread making process: in the first step a sponge is made and allowed to ferment for a period of time (prefermenting), and in the second step the sponge is combined with further ingredients thus creating a total formula dough. After development of the dough the baked product is baked.

Flour brew, also referred to as liquid sponge herein, is one type of preferment methods in the sponge and dough process. It needs more water added compared with traditional sponge, in which usually a little more water than flour. It usually contains about 10-50% of the total dough flour and ferments in a tank for one or two hours. Flour brew has the advantage of being pumpable compared with sponge and dough method, especially in the production of hamburger buns. Due to agitation in the fermentation tank, gluten may separate forming strings that will cause clog in the pump an heat exchanger later in the process, what is undesired. Currently ADA is used to stabilize a liquid sponge and to avoid gluten separation also called gluten wash. There is a need to replace ADA.

The term tortilla herein includes corn tortilla and wheat tortilla. A corn tortilla is a type of thin, flat bread, usually unleavened made from finely ground maize (usually called “corn” in the United States). A flour tortilla is a type of thin, flat bread, usually unleavened, made from finely ground wheat flour. The term tortilla further includes a similar bread from South America called arepa, though arepas are typically much thicker than tortillas. The term tortilla further includes a laobing, a pizza-shaped thick “pancake” from China and an Indian Roti, which is made essentially from wheat flour. A tortilla usually has a round or oval shape and may vary in diameter from about 6 to over 30 cm.

Baked products obtained by using a dough prepared according to the invention are also encompassed by the present invention. All the embodiments which were mentioned above for the flour, with all the preferences mentioned above, also apply to doughs and baked products according to the invention.

In yet another aspect, compositions according to the invention may be used as improving agents, also referred to as bread improvers or dough improvers, which are combined with flour to improve functionalities of the bread, dough or of the baked product made from the dough. The improvement is in comparison to a dough which does not contain a bread or dough improver according to the invention and may be reflected in any functionality of the bread, dough or baked product from the dough, such as increased strength of the dough, increased viscoelasticity of the dough, increased stability of the dough, reduced stickiness of the dough, improved extensibility of the dough, improved machinability of the dough. increased volume of the baked product, improved flavour of the baked product, improved crumb structure of the baked product, improved crumb softness of the baked product, reduced blistering of the baked product, improved crispiness, improved resilience both initial and in particular after storage, reduced hardness after storage or improved anti-staling properties of the baked product.

The term “increased strength of the dough” is defined herein as the property of a dough that has generally more viscoelastic properties and/or requires more work input to mould and shape.

The term “increased viscoelasticity of the dough” is defined herein as the property of a dough which has a higher tendency to regain its original shape after being subjected to a certain physical strain.

The term “increased stability of the dough” is defined herein as the property of a dough that is less susceptible to forming faults as a consequence of mechanical abuse thus better maintaining its shape and volume and is evaluated by the ratio of height:width of a cross section of a loaf after normal and/or extended proof.

The term “reduced stickiness of the dough” is defined herein as the property of a dough that has less tendency to adhere to surfaces, e.g., in the dough production machinery, and is either evaluated empirically by the skilled test baker or measured by a suitable system known in the art, such as Warburtons dough stickiness system. Reduced dough stickiness, also referred to herein as reduced stickiness of the dough, may be determined by methods known in the art, such as by evaluation by a skilled baker or by using a texture analyser. In one embodiment, reduced dough stickiness is determined in a Warburtons dough stickiness system. Preferably dough stickiness is determined at ambient temperature. Ambient temperature may typically be between 20 and 30 degrees Celcius.

Dough stickiness may be evaluated through tactile assessment for example while shaping the dough. Alternatively dough stickiness may be evaluated after shaping the dough, by touching the surface of the shaped dough and sensing how much it sticks to the fingers. If the dough sticks less to the fingers than a reference it is evaluated as having a reduced dough stickiness. Alternatively dough stickiness may be evaluated after cutting the dough, by touching the surface of the freshly cut dough surface and sensing how much it sticks to the fingers. If the dough sticks less to the fingers than a reference it is evaluated as having a reduced dough stickiness.

The term “improved extensibility of the dough” is defined herein as the property of a dough that can be subjected to increased strain or stretching without rupture. The term “improved machinability of the dough” is defined herein as the property of a dough that is generally less sticky and/or more firm and/or more elastic. Consequently there is less fouling of plant equipment and a reduced need for cleaning.

The term “improved crumb structure of the baked product” is defined herein as the property of a baked product with finer cells and/or thinner cell walls in the crumb and/or more uniform/homogenous distribution of cells in the crumb and is usually evaluated visually by the baker or by digital image analysis as known in the art (eg. C-cell, Calibre Control International Ltd, Appleton, Warrington, UK).

The term “improved softness of the baked product” is the opposite of “hardness” and is defined herein as the property of a baked product that is more easily compressed and is evaluated either empirically by the skilled test baker or measured by the use of a texture analyzer known in the art.

The term “reduced blistering of the baked product” is defined herein as a visually determined reduction of blistering on the crust of the baked bread.

The term “improved crispiness” is defined herein as the property of a baked product to give a crispier sensation than a reference product as known in the art, as well as to maintain this crispier perception for a longer time than a reference product. This property can be quantified by measuring a force versus distance curve at a fixed speed in a compression experiment using e.g. a texture analyzer TA-XT Plus (Stable Micro Systems Ltd, Surrey, UK), and obtaining physical parameters from this compression curve, viz. (i) force of the first peak, (ii) distance of the first peak, (iii) the initial slope, (iv) the force of the highest peak, (v) the area under the graph and (vi) the amount of fracture events (force drops larger than a certain preset value). Indications of improved crispness are a higher force of the first peak, a shorter distance of the first peak, a higher initial slope, a higher force of the highest peak, higher area under the graph and a larger number of fracture events. A crispier product should score statistically significantly better on at least two of these parameters as compared to a reference product. In the art, “crispiness” is also referred to as crispness, crunchiness or crustiness, meaning a material with a crispy, crunchy or crusty fracture behaviour.

The term “improved anti-staling properties of the baked product” is defined herein as the properties of a baked product that have a reduced rate of deterioration of quality parameters, e.g. reduced hardness after storage and/or decreased loss of resilience after storage. Anti-staling properties may be demonstrated by a reduced hardness after storage of the baked product. The enzyme composition according to the invention or the pre-mix according to the invention may result in reduced hardness, e.g. in a baked product that is more easily compressed. The hardness of the baked product may be evaluated either empirically by the skilled test baker or measured by the use of a texture analyzer known in the art. The hardness measured within 24 hours after baking is called initial hardness. The hardness measured 24 hours or more after baking is called hardness after storage, and is also a measure for determining shelf life. In case the initial hardness has reduced, it has improved. In case the hardness after storage has reduced, it has improved. Resilience of the baked product is preferably measured by the use of a texture analyzer known in the art. The resilience measured within 24 hours after baking is called initial resilience. The resilience measured 24 hours or more after baking is called resilience after storage, and is also a measure for determining shelf life. Freshly baked product typically gives crumb of high initial resilience but resilience is lost over shelf-life. Improved anti-staling properties may be demonstrated by a reduced loss of resilience over storage.

Improved shock resistance of a dough may be demonstrated as follows.

Bread tins filled with proofed dough are dropped from a certain drop-height, e.g. 9 cm height, just before they enter the oven to be baked. This may be done by simultaneously pulling away 2 blocks having this drop-height from below the bottom of the tin. This way the tin drops over the drop-height and the dough experiences a shock. The shock resistance of a dough is improved if, after baking the dough, the volume of the loaf is larger as compared to a reference loaf (which may also be called a control) loaf and/or if the hardness of the loaf after baking the dough is lower as compared to a reference loaf (which may also be called a control).

A use of the invention includes the use of Penicillium glucose oxidase, a pre-mix comprising Penicillium glucose oxidase, a flour comprising Penicillium glucose oxidase or a composition comprising a Penicillium glucose oxidase to improve shock resistance of a dough as compared to a reference dough.

A use of the invention includes the use of Penicillium glucose oxidase, a pre-mix comprising Penicillium glucose oxidase, a flour comprising Penicillium glucose oxidase or a composition comprising a Penicillium glucose oxidase to improve shock resistance of a dough as compared to a reference dough, wherein the reference dough does not comprise Penicillium glucose oxidase.

A use of the invention includes the use of Penicillium glucose oxidase, a pre-mix comprising Penicillium glucose oxidase, a flour comprising Penicillium glucose oxidase or a composition comprising a Penicillium glucose oxidase to improve shock resistance of a dough as compared to a reference dough, wherein the reference dough comprises a chemical oxidizer such as ADA.

A use of the invention includes the use of Penicillium glucose oxidase, a pre-mix comprising Penicillium glucose oxidase, a flour comprising Penicillium glucose oxidase or a composition comprising a Penicillium glucose oxidase to improve shock resistance of a dough as compared to a reference dough, wherein the reference dough comprises Aspergillus glucose oxidase.

In an aspect of the invention, a dough of the invention is a dough comprising Penicillium glucose oxidase having an improved shock resistance as compared to a reference dough.

In an aspect of the invention, a dough of the invention is a dough comprising Penicillium glucose oxidase having an improved shock resistance as compared to a reference dough, wherein the reference dough does not comprise Penicillium glucose oxidase.

In an aspect of the invention, a dough of the invention is a dough comprising Penicillium glucose oxidase having an improved shock resistance as compared to a reference dough, wherein the reference dough comprises a chemical oxidizer such as ADA.

In an aspect of the invention, a dough of the invention is a dough comprising Penicillium glucose oxidase having an improved shock resistance as compared to a reference dough, wherein the reference dough comprises Aspergillus glucose oxidase.

The invention further relates to a method to stabilize a liquid sponge, the method comprising the step of adding a Penicillium glucose oxidase to at least one dough ingredient.

In an aspect of the method to stabilize a liquid sponge according to the invention, in addition to the Penicillium glucose oxidase an Amyloglucosidase and/or ascorbic acid are added.

The invention further relates to the use of a composition comprising a Penicillium glucose oxidase to stabilize a liquid sponge.

In an aspect of the use according to the invention, said use is the use of a composition comprising a Penicillium glucose oxidase to replace ADA as stabilizer of a liquid sponge.

In an aspect of the use according to the invention, said use is the use of a composition comprising a Penicillium glucose oxidase and an Amyloglucosidase and/or ascorbic acid to stabilize a liquid sponge.

In an aspect of the use according to the invention, said use is the use of a composition comprising Penicillium glucose oxidase and an Amyloglucosidase and/or ascorbic acid to replace ADA as stabilizer of a liquid sponge.

In aspect 1 of the invention, a use of the invention is the use of a composition comprising a Penicillium glucose oxidase as a chemical oxidizer replacement in the baking industry.
In aspect 1A of the invention a use of the invention is the use of a composition comprising a Penicillium glucose oxidase, preferably as set out in amino acids amino acids 19-604 of SEQ ID No.1, to replace a chemical oxidizer in the production of a baked product.
In aspect 1B of the invention a use of the invention is the use of a composition comprising a Penicillium glucose oxidase, preferably as set out in amino acids amino acids 19-604 of SEQ ID No.1, to replace azodicarbonamide in the production of a baked product.
In aspect 1C of the invention a use of the invention is the use of a composition comprising a Penicillium glucose oxidase, preferably as set out in amino acids amino acids 19-604 of SEQ ID No.1, to replace potassium bromate in the production of a baked product.
In aspect 1D of the invention a use of the invention is the use of a composition comprising a Penicillium glucose oxidase, preferably as set out in amino acids 19-604 of SEQ ID No.1, to fully replace a chemical oxidizer in the production of a baked product. In aspect 1E of the invention a use of the invention is the use of a composition comprising a Penicillium glucose oxidase, preferably as set out in amino acids 19-604 of SEQ ID No.1, to fully replace azodicarbonamide in the production of a baked product.
In aspect 1F of the invention a use of the invention is the use of a composition comprising a Penicillium glucose oxidase, preferably as set out in amino acids 19-604 of SEQ ID No.1, to fully replace potassium bromate in the production of a baked product.
In aspect 1G of the invention a use of the invention is the use of a Penicillium glucose oxidase, preferably as set out in amino acids 19-604 of SEQ ID No.1, to replace a chemical oxidizer in the preparation of a baked product.
In aspect 1H of the invention a use of the invention in the use of a Penicillium glucose oxidase, preferably as set out in amino acids 19-604 of SEQ ID No.1, to replace azodicarbonamide in the preparation of a baked product.
In aspect 1G of the invention a use of the invention in the use of a Penicillium glucose oxidase, preferably as set out in amino acids 19-604 of SEQ ID No.1, to replace potassium bromate in the preparation of a baked product.
In aspect 2 of the invention, a use of the invention is the use according to aspect 1, wherein the composition further comprises ascorbic acid.
In aspect 2A of the invention, a use of the invention is the use according to any one of aspects 1A to 1G, wherein the composition further comprises ascorbic acid.
In aspect 3 of the invention, a use of the invention is the use according to aspect 1 or aspect 2, wherein the composition further comprises at least one of amyloglycosidase, bacterial or fungal hemicellulase and bacterial or fungal alpha amylase.
In aspect 3A of the invention, a use of the invention is the use according to any one of aspects 1A to 1G, wherein the composition further comprises at least one of amyloglucosidase, bacterial or fungal hemicellulase and bacterial or fungal alpha amylase.
In aspect 4 of the invention, a use of the invention is the use according to any one of aspects 1 to 3, wherein the glucose oxidase has an amino acid sequence having at least 75% identity to amino acids 19-604 of SEQ ID No. 1.
In aspect 4A of the invention, a use of the invention is the use according to any one of aspects 1A to 1G, wherein the glucose oxidase has an amino acid sequence having at least 75% identity to amino acids 19-604 of SEQ ID No. 1.
In aspect 5 of the invention, a use of the invention is the use according to any one of aspects 1 to 4, wherein the glucose oxidase completely or partially replaces bromate.
In aspect 5A of the invention, a use of the invention is the use according to any one of aspects 1A to 1G, wherein the glucose oxidase completely or partially replaces bromate.
In aspect 6 of the invention, a use of the invention is the use according to any one of aspects 1 to 5, wherein the glucose oxidase completely or partially replaces azodicarbonamide (ADA).
In aspect 6A of the invention, a use of the invention is the use according to any one of aspects 1A to 1G, wherein the glucose oxidase completely or partially replaces azodicarbonamide (ADA).
In aspect 7 of the invention, a pre-mix of the invention is a pre-mix comprising
a) a glucose oxidase with an amino acid sequence having at least 75% identity to amino acids 19-604 of SEQ ID No.1;
b) 0 to 20 ppm azodicarbonamide; and
c) 0 to 30 ppm bromate.
In aspect 7A of the invention, a pre-mix of the invention is a pre-mix comprising
a) a glucose oxidase with an amino acid sequence having at least 75% identity to amino acids 19-604 of SEQ ID No.1;
b) 0 to 20 ppm azodicarbonamide based on flour weight; and
c) 0 to 30 ppm bromate based on flour weight.
In aspect 8 of the invention, a method of the invention is a method for improving flour, which method comprises adding to the flour by weight of flour 1 to 50 ppm of a Penicillium glucose oxidase having an activity in the range of 2500-4000 glucose oxidase units/gram enzyme.
In aspect 9 of the invention, a flour of the invention is flour comprising by weight of flour
a) 1-50 ppm of a glucose oxidase with an amino acid sequence having at least 75% identity to amino acids 19-604 of SEQ ID No.1;
b) 0 to 20 ppm azodicarbonamide; and
c) 0 to 30 ppm bromate.
In aspect 10 of the invention, a flour of the invention is flour according to aspects 8 or 9 comprising by weight of flour
a) 1-50 ppm of a glucose oxidase with an amino acid sequence having at least 75% identity to amino acids 19-604 of SEQ ID No.1;
b) 10-50 ppm ascorbic acid;
c) 0-20 ppm amyloglucosidase;
d) 0 to 20 ppm azodicarbonamide; and
e) 0 to 30 ppm bromate.
In aspect 11 of the invention, a flour of the invention is flour according to any one of aspects 8 to 10 comprising by weight of flour
a) 1-50 ppm of a glucose oxidase with an amino acid sequence having at least 75% identity to amino acids 19-604 of SEQ ID No.1;
b) 10-50 ppm ascorbic acid;
c) 1-20 ppm fungal alpha amylase;
d) 1-50 ppm hemicellulose;
e) 0 to 20 ppm azodicarbonamide; and
f) 0 to 30 ppm bromate.
In aspect 11A of the invention, a flour of the invention is flour according to any one of aspects 8 to 10 comprising by weight of flour
a) 1-50 ppm of a glucose oxidase comprising an amino acid sequence having at least 75% identity to amino acids 19-604 of SEQ ID No. 1;
b) 10-50 ppm ascorbic acid;
c) 1-20 ppm fungal alpha amylase;
d) 1-50 ppm hemicellulase;
e) 0 to 20 ppm azodicarbonamide; and
f) 0 to 30 ppm bromate.
In aspect 12 of the invention, a method of the invention is a method for preparing a dough comprising combining a pre-mix according to aspect 7 or a flour according to any one of aspects 8 to 11 with at least one dough ingredient.
In aspect 12A of the invention, a method of the invention is a method for preparing a dough comprising combining a pre-mix according to aspect 7A or a flour according to aspect 11A with at least one dough ingredient.
In aspect 13 of the invention, a dough of the invention is a dough prepared from a pre-mix according to aspect 7 or from a flour according to any one of aspects 8 to 11.
In aspect 13A of the invention, a dough of the invention is a dough prepared from a pre-mix according to aspect 7A or from a flour according to aspect 11A.
In aspect 14 of the invention, a method of the invention is a method for preparing a baked product comprising the step of baking the dough according to aspect 13.
In aspect 14A of the invention, a method of the invention is a method for preparing a baked product comprising the step of baking the dough according to aspect 13A.
In aspect 15 of the invention, a baked product of the invention is a baked product prepared from a pre-mix according to aspect 7, from a flour according to any one of aspects 8 to 11, or a dough according to aspect 13.
In aspect 15A of the invention, a baked product of the invention is a baked product prepared from a pre-mix according to aspect 7A, from a flour according to aspect 11A, or a dough according to aspect 13A.
In aspect 16 of the invention, a use of the invention is the use of Penicillium glucose oxidase, a pre-mix comprising Penicillium glucose oxidase, a flour comprising Penicillium glucose oxidase or a composition comprising a Penicillium glucose oxidase to improve shock resistance of a dough.
In aspect 17 of the invention, a dough of the invention is a dough comprising Penicillium glucose oxidase and having an improved shock resistance as compared to a reference dough.
In aspect 18 of the invention, the invention relates to a method to replace a chemical oxidiser in a baking product comprising the step of adding Penicillium glucose oxidase, preferably as set out in amino acids 19-604 of SEQ ID No.1, as an ingredient in the preparation of said product.
In aspect 19 of the invention, the invention relates to a method to replace a chemical oxidiser in a baking product comprising the step of adding Penicillium glucose oxidase, preferably as set out in amino acids 19-604 of SEQ ID No.1, to at least one dough ingredient.
In aspect 19A of the invention, the invention relates to a method according to aspect 19, wherein the Penicillium glucose oxidase is part of a composition further comprising a dough ingredient and/or an additional enzyme.
In aspect 19B of the invention, the invention relates to a method according to aspect 19, wherein Penicillium glucose oxidase is part of a composition further comprising one or more components selected from the group consisting of milk powder, gluten, granulated fat, an additional enzyme, an amino acid, a salt, an oxidant such as ascorbic acid, a reducing agent such as L-cysteine, an emulsifier such as lecithine, mono-glycerides, di-glycerides, glycerol monostearate, sodium stearoyl lactylate, calcium stearoyl lactylate, polyglycerol esters of fatty acids and diacetyl tartaric acid esters of mono- and diglycerides, gums such as guar gum and xanthan gum, flavours, acids such as citric acid and propionic acid, starch, modified starch, gluten, humectants such as glycerol, and preservatives.
In aspect 20 of the invention, the invention relates to a method to replace a chemical oxidiser in a baking product comprising the step of adding a composition comprising Penicillium glucose oxidase, preferably as set out in amino acids 19-604 of SEQ ID No.1, as an ingredient in the preparation of said product.
In aspect 21 of the invention, the invention relates to a method to replace a chemical oxidiser in a baking product comprising the step of adding a composition comprising Penicillium glucose oxidase, preferably as set out in amino acids 19-604 of SEQ ID No.1, to at least one dough ingredient.
In aspect 22 of the invention, a method of the invention is the method according to aspect 19, wherein the at least one dough ingredient comprises ascorbic acid.
In aspect 22 of the invention, a method of the invention is the method according to any one of aspect 20 or 21, wherein the composition further comprises ascorbic acid.
In aspect 23 of the invention, a method of the invention is the method according to aspect 19, wherein the at least one dough ingredient comprises at least one of amyloglucosidase, bacterial or fungal hemicellulase, and bacterial or fungal alpha amylase.
In aspect 24 of the invention, a method of the invention is the method according to any one of aspects 20 or 21, wherein the composition further comprises amyloglucosidase, bacterial or fungal hemicellulase, and bacterial or fungal alpha amylase.
In aspect 25 of the invention, a method of the invention is the method according to any one of aspects 18 to 23, wherein the glucose oxidase comprises an amino acid sequence having at least 75% identity to amino acids 19-604 of SEQ ID No. 1.
In aspect 26 of the invention, a method of the invention is the method according to any one of aspects 18 to 25, wherein the glucose oxidase completely or partially replaces bromate.
In aspect 27 of the invention, a method of the invention is the method according to any one of aspects 18 to 25, wherein the glucose oxidase completely or partially replaces azodicarbonamide (ADA).
In aspect 28 of the invention, a method of the invention is the method according to any one of aspects 18 to 27, wherein the method comprises the step of adding by weight of flour
a) 1-50 ppm of a glucose oxidase with an amino acid sequence having at least 75% identity to amino acids 19-604 of SEQ ID No.1;
b) 10-50 ppm ascorbic acid;
c) 1-20 ppm fungal alpha amylase;
d) 1-50 ppm hemicellulase; and to flour.

In aspect 29 of the invention, a method of the invention is the method according to any one of aspects 18 to 27, wherein the method comprises the step of adding by weight of flour:

a) 1-50 ppm of a glucose oxidase with an amino acid sequence having at least 75% identity to amino acids 19-604 of SEQ ID No.1, preferably a glucose oxidase as set out in amino acids 19-604 of SEQ ID No.1;

b) 10-50 ppm ascorbic acid; and

c) 1-30 ppm Amyloglucosidase

to flour.

Adding by weight of flour herein means adding by weight of the total amount of flour used in the recipe. For the American Style sponge and dough method this means the sum of the flour used in the liquid sponge recipe and in the dough recipe.

In aspect 30 a use according to the invention is a use according to aspect 1, wherein said composition is used to stabilize a liquid sponge in the production of a baked product.

### EXAMPLES

Materials and Methods

Flour Corrector

The flour corrector used was a composition comprising 30 ppm ascorbic acid (from DSM Nutritional Products, Switzerland), 2 ppm Bakezyme® P500 (fungal alpha-amylase from DSM, The Netherlands), 15 ppm Bakezyme® HSP6000 (fungal hemicellulase from DSM, The Netherlands) and Kolibri flour (Meneba, the Netherlands) as filling material.

Oxidizers

Potassium bromate was obtained from Alpha Aesar, U.S.A., Azodicarbonamide (ADA) was obtained from AB Mauri BTC, The Netherlands Ascorbic acid was obtained from DSM, Nutritional Products, Switzerland;

Penicillium glucose oxidase was Penicillium chrysogenum glucose oxidase with a protein sequence as depicted in SEQ ID No.1 was prepared by transformation of an Aspergillus with a construct comprising a cDNA sequence as depicted in SEQ ID No. 2.

The Penicillium glucose oxidase used was Penicillium chrysogenum glucose oxidase with a protein sequence as depicted in SEQ ID No.1 prepared by transformation of an Aspergillus with a construct comprising a cDNA sequence as depicted in SEQ ID No. 2 and subsequent expression of the enzyme.

BakeZyme® GO10.000 BG, (Aspergillus glucose oxidase, from DSM Food Specialties, the Netherlands) was used.

For comparison, BakeZyme® GO10.000 BG, (Aspergillus tubingensis glucose oxidase, from DSM Food Specialties, the Netherlands) was used.

Determination of Glucose Oxidase Activity

Glucose oxidase activity was determined in an assay in which gluconic acid formed is titrated. Thereto, 1 ml of diluted glucose oxidase was added to 25 ml of preheated 30 g/I glucose monohydrate solution at 35 degrees C. Sample dilutions and substrate were prepared in 50 mM HAc buffer at pH 5.1, containing 50 mM NaCl. After 15 minutes incubation at 35 degrees C., the reaction was terminated by the addition of 10 ml 0.1 N NaOH, at the same time neutralizing the gluconic acid formed. Excess NaOH was titrated with 0.05 M HCl. The difference in HCl consumption between a sample and blank run is a measure for the amount of glucose oxidase activity. One glucose oxidase unit is defined as the amount of enzyme needed to oxidize 3 mg of glucose to gluconic acid under conditions of the assay.

Activity of Penicillium glucose oxidase used was 3300 glucose oxidase units/gram enzyme. Ppm means mg/kg, e.g. 20 ppm means 20 mg of the indicated product per kg flour.

Determination of Hardness of Bread

The hardness of bread slices was measured using a Texture Analyser TA-XTPlus from Stable Micro Systems apparatus and applying the following settings.

Settings
Test mode=Compression
Pre-test speed=3 mm/s
Test speed=1 mm/s
Post test speed 5 mm/s
Distance=5 mm
Hold time=10 sec
Trigger force=5 g

The bread was sliced with a bread slicer set at 2.1 cm slice distance.

The hardness listed is the Force measured; the max peak value recorded in gram. Resilience is the Force (F) after 10 sec holding time (F2) divided by max peak force (F1) multiplied by 100. Resilience=(F2/F1)×100

### Example 1 Bromate Replacement in Bread

The performance of Penicillium glucose oxidase as a bromate replacer in bread was examined in a direct floor bread baking process and in an overnight bread baking process.

Bread doughs were prepared by mixing the ingredients listed in Table 1 together with bromate or bromate replacement according to Table 2 first column in a Diosna SP-12 mixer, 400 turns, at a frequency of 25 Hz and thereafter 70 Wh at a frequency of 50 Hz, to a final dough temperature of 27° C. The dough was divided in 8 pieces of 350 g, rounded and proofed in the bench proof cabinet for 20 minutes at 28° C. and 90% relative humidity. Then, the dough pieces were moulded using a Bertrand baguette moulder and divided in two groups of four dough pieces.

TABLE 1
Basic bread recipe

| Ingredient | grams | % | Type |
|---|---|---|---|
| Flour | 2000 | 100 | EPI B, Paniflower, Belgium |
| Salt | 36 | 1.8 | Suprasel, Akzo Nobel, the Netherlands |
| Fresh yeast | 50 | 2.5 | Koningsgist, AB Mauri, the Netherlands |
| Flour corrector | 10 | 0.5 | See materials & methods |
| Water | 1240 | 62 | ⁺/−1% |

For a direct floor bread baking process, four dough pieces were placed on a sheeter. They were proofed in a Wachtel Octopus proof cabinet for 120 minutes at 32° C. with a relative humidity of 88%. Then, the fully proofed dough pieces were placed in a Wachtel Piccolo oven and baked in a first phase at 245° C. top heat and 235° C. floor heat for 5 minutes with initial steam addition. After that the temperature was decreased to 225° C. top heat and 235° C. floor heat for 20 minutes in a second phase.

For an overnight bread baking process, four dough pieces were placed on greased baking trays. They were stored in a Wachtel Octopus proof cabinet at 2° C. with a relative humidity of 88% for 18 hrs. After that, the dough pieces were proofed in a Wachtel Octopus proof cabinet for 180 minutes at 21° C. with a relative humidity of 88%. The fully proofed dough pieces were placed in a Wachtel Comet rotation oven and baked in a first phase at 260° C. for 5 minutes with initial steam addition. After that the temperature was decreased to 230° C. for 15 minutes in a second phase.

After baking, the oven was unloaded, the breads were taken off the baking trays and placed on a rack to cool for at least 1 hour at ambient temperature, which was typically between 20 and 25° C. After 1-2 hours cooling, the breads were assessed on volume, shape and structure.

After cooling down to room temperature, the volumes of the loaves were determined by an automated bread volume analyser (BVM-3, TexVol Instruments). The loaf volume of the control bread is defined as 100%. Results are shown in Table 2, which shows the average value of four loaves for each recipe. The control refers to a loaf of bread prepared from the ingredients in Table 1, i.e. to which no bromate, Penicillium glucose oxidase, ascorbic acid or amyloglucosidase was added.

TABLE 2
Average volume of four loaves

| | Relative Volume | Relative Volume |
|---|---|---|
| | (%) direct | (%) overnight |
| Control | 100 | 100 |
| 20 ppm Bromate | 117 | 123 |
| 30 ppm Bromate | 116 | 121 |
| 40 ppm Bromate | 112 | 124 |
| 10 ppm Penicillium glucose | 105 | 110 |
| oxidase | | |
| 20 ppm Penicillium glucose | 105 | 110 |
| oxidase | | |
| 10 ppm Penicillium glucose | 117 | 117 |
| oxidase and 20 ppm Ascorbic | | |
| acid | | |
| 20 ppm Penicillium glucose | 107 | 116 |
| oxidase and 40 ppm Ascorbic | | |
| Acid | | |
| 20 ppm Penicillium glucose | 122 | 118 |
| oxidase and 40 ppm Ascorbic | | |
| Acid and 5 ppm Bakezyme | | |
| AG1100 | | |
| 20 ppm Penicillium glucose | 127 | 121 |
| oxidase and 40 ppm Ascorbic | | |
| Acid and 10 ppm Bakezyme | | |
| AG1100 | | |

These results show a 5-10% increase in volume compared to the control, if Penicillium glucose oxidase alone is used to replace bromate. This increase is observed both in the direct process and the overnight process. This indicates an increase in dough and/or fermentation stability.

These results show a more substantial increase in volume, equivalent to fermentation stability, if the Penicillium glucose oxidase is added in combination with ascorbic acid.

The addition of small dosages of amyloglucosidase, which is a substrate provider for glucose oxidase, increases the volume of the final bread even further. The volume increase obtained is equivalent to (overnight process) or better than (direct process) the volume increase obtained when using bromate, indicating a further improvement in dough and/or fermentation stability.

The consistency, body, development, extensibility, elasticity and stickiness of the dough were evaluated by an experienced baker. Dough containing 20 ppm Penicillium glucose oxidase and 40 ppm ascorbic acid and 10 ppm Bakezyme AG1100 was considered to be equivalent to dough containing 30 ppm bromate. Crumb structure and crumb colour of the bread were evaluated as good.

This shows that bromate can successfully be replaced by a Penicillium glucose oxidase, a Penicillium glucose oxidase and ascorbic acid or by a Penicillium glucose oxidase/ascorbic acid/amyloglucosidase.

### Example 2 Azodicarbonamide Replacement in Bread

The performance of Penicillium glucose oxidase as an azodicarbonamide (ADA) replacer in bread was examined in tin bread using an American style direct open top bread making process.

The ingredients listed in Table 3 were mixed in a Diosna SP-12 mixer, 400 turns at a frequency of 25 Hz and thereafter 108 Wh at a frequency of 50 Hz, to a final dough temperature of 28° C.

TABLE 3
Basic bread recipe

| Ingredient | grams | % | Type |
|---|---|---|---|
| Flour | 3000 | 100 | BG100, Paniflower, Belgium |
| Salt | 60 | 2 | Suprasel, Akzo Nobel, the |
| | | | Netherlands |
| Instant dry yeast | 30 | 1 | Fermipan brown, AB Mauri, the |
| | | | Netherlands |
| Fresh yeast | 30 | 1 | Koningsgist, AB Mauri, the |
| | | | Netherlands |
| Sugar | 240 | 8 | Castor, Suiker Unie, the |
| | | | Netherlands |
| Oil | 75 | 2.5 | Rapeseed, Aldoc, the Netherlands |
| Fat | 75 | 2.5 | Soft Bakers fat, CSM Benelux. |
| SSL | 6 | 0.2 | Prefera 6000, BASF, Germany. |
| GMS | 6 | 0.2 | Nutrisoft 55, BASF, Germany. |
| Calcium | 10 | 0.35 | Probake, Kemira, the Netherlands |
| propionate | | | |
| Ascorbic Acid | 20 ppm | | DSM Nutritional Products, |
| | | | Switzerland |
| BakeZyme P500 | 5 ppm | | Fungal Alpha - amylase, DSM |
| | | | Food Specialties, the Netherlands |
| BakeZyme | 15 ppm | | Fungal Hemicellulase, DSM |
| HSP6000 | | | Food Specialties, the Netherlands |
| BakeZyme | 15 ppm | | Bacterial Hemicellulase, DSM |
| BXP500* | | | Food Specialties, the Netherlands |
| Water | 1800 | 60 | ⁺/−1% |

Then, the dough was cold extruded at a Bear Varimixer meat extruder, which subjects the dough to high shear and which is equivalent to handling dough with a dough pump at industrial scale The dough was divided in 8 pieces of 350 g, rounded and proofed at the table for 10 minutes at room temperature. Subsequently, the dough pieces were moulded using a Glimek MO-671 moulder, placed in greased tins and proofed in a Wachtel Octopus proof cabinet for 60 minutes at 40° C. with a relative humidity of 88%. The fully proofed dough pieces were placed in a Wachtel Comet rotation oven and baked first phase at 205° C. for 10 minutes with initial steam addition. After that, the temperature was decreased to 180° C. for 15 minutes, second phase. After baking, the oven was unloaded, the breads were de-panned and placed on a rack to cool for at least 1 hour at ambient temperature, which was typically between 20 and 25° C. After 1-2 hours cooling, the breads were assessed on volume, shape, structure and softness.

After cooling down to room temperature the volumes of the loaves were determined by an automated bread volume analyser (BVM-3, TexVol Instruments). The loaf volume of the control bread was set at 100%. After 1 day, bread hardness was measured as described in Materials and Methods. The bread was sliced with a bread slicer set at 2.1 cm slice distance. Due to test set-up, results should be evaluated per pair (e.g. control vs. 40 ppm ADA, and 10 ppm vs. 40 ppm Penicillium glucose oxidase etc.). Results for volume and hardness are presented in Table 4. The control refers to a loaf of bread prepared from the ingredients in Table 3 and to which no ADA or Penicillium glucose oxidase was added.

TABLE 4
Average hardness values of four loaves

| | | | Hardness |
|---|---|---|---|
| test | | Volume (ml) | day 1 (g) |
| 1a | Control | 1359 | 258 |
| 1b | 40 ppm ADA | 1437 | 198 |
| 2a | 10 ppm Penicillium glucose oxidase | 1415 | 218 |
| 2b | 40 ppm Penicillium glucose oxidase | 1506 | 201 |
| 3a | 40 ppm ADA | 1301 | 200 |
| 3b | 20 ppm Penicillium glucose oxidase | 1302 | 234 |

These results show that the loaf volume is increasing with increasing dosage of Penicillium glucose oxidase. Hence, the effect of Penicillium glucose oxidase is dose-dependent. The results also show that 20 ppm Penicillium glucose oxidase gives an improvement in volume, compared to the control, which is equivalent to that of 40 ppm ADA. ADA is known for improving dough stability or dough resistance, which is important in baked products which are produced using a process in which the dough is subjected to high shear. Limited dough stability results in an open cell structure and would have resulted in a lack of volume, which is not observed here. The results show that

Penicillium glucose oxidase may successfully be used as an ADA replacer, even in doughs which are subjected to high shear, because there is no loss of volume upon replacement.

The results on hardness show that bread hardness is decreasing with increasing dosage of Penicillium glucose oxidase. Decrease in hardness at day 1 is an indication for improved crumb structure.

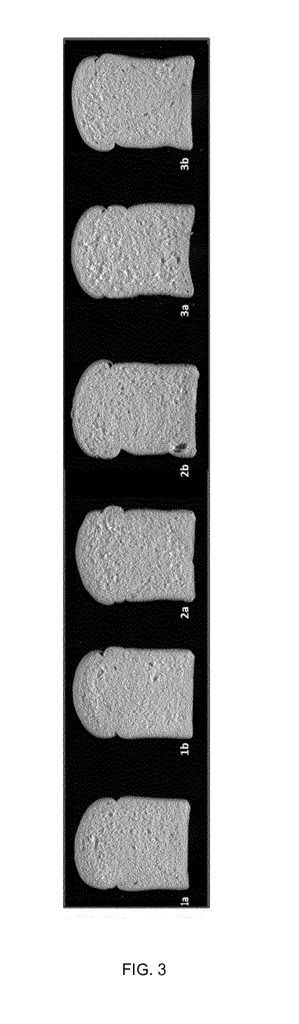

Shape and crumb structure of the bread were determined by the C-Cell structure analyser (Calibre, United Kingdom). Results are illustrated in FIG. 3. The results show that the fineness of the crumb structure is increasing with increasing dosage of Penicillium glucose oxidase.

These results illustrate that ADA in baking processes can successfully be replaced by a Penicillium glucose oxidase.

### Example 3 Azodicarbonamide Replacement in Bread

The performance of Penicillium glucose oxidase as an azodicarbonamide (ADA) replacer in bread was examined in tin bread using an American style direct open top bread making process under the conditions as described in Example 2 herein, with the difference of leaving out ascorbic acid.

The ingredients as listed in table 5 were used.

TABLE 5
Basic bread recipe

| Ingredient | grams | % | Type |
|---|---|---|---|
| Flour | 3000 | 100 | BG100, Paniflower, Belgium |
| Salt | 60 | 2 | Suprasel, Akzo Nobel, the |
| | | | Netherlands |
| Instant dry yeast | 30 | 1 | Fermipan brown, AB Mauri, the |
| | | | Netherlands |
| Fresh yeast | 30 | 1 | Koningsgist, AB Mauri, the |
| | | | Netherlands |
| Sugar | 240 | 8 | Castor, Suiker Unie, the |
| | | | Netherlands |
| Oil | 75 | 2.5 | Rapeseed, Aldoc, the Netherlands |
| Fat | 75 | 2.5 | Soft Bakers fat, CSM Benelux. |
| SSL | 6 | 0.2 | Prefera 6000, BASF, Germany. |
| GMS | 6 | 0.2 | Nutrisoft 55, BASF, Germany. |
| Calcium | 10 | 0.35 | Probake, Kemira, the Netherlands |
| propionate | | | |
| BakeZyme P500 | 5 ppm | | Fungal Alpha - amylase, DSM |
| | | | Food Specialties, the Netherlands |
| BakeZyme | 15 ppm | | Fungal Hemicellulase, DSM |
| HSP6000 | | | Food Specialties, the Netherlands |
| BakeZyme | 15 ppm | | Bacterial Hemicellulase, DSM |
| BXP500* | | | Food Specialties, the Netherlands |
| Water | 1800 | 60 | ⁺/−1% |

Results are listed in Table 6, whereby the hardness was measured at day 3 (with day 1 being the day that the bread was baked). The control refers to a loaf of bread prepared from the ingredients in Table 5 and to which no ADA, no ascorbic acid, no Penicillium glucose oxidase and no Aspergillus glucose oxidase was added.

The result indicated with “shock” in Table 6 were determined as follows: the bread tins filled with proofed dough were dropped from 9 cm height just before they entered the oven. This was done by simultaneously pulling away 2 blocks of 9 cm height from below the bottom of the tin, this way the tin dropped 9 cm and the dough experienced a shock.

TABLE 6
Average hardness values of four loaves

| | | | | | Hardness |
|---|---|---|---|---|---|
| | | Volume | Volume | Hardness | day 3 (g) |
| est | | (ml) | (ml) shock | day 3 (g) | shock |
| a | Control | 1205 | 1178 | 376 | 370 |
| b | 40 ppm ADA | 1096 | 1103 | 352 | 351 |
| a | 20 ppm Penicillium | 1293 | 1247 | 277 | 292 |
| | glucose oxidase | | | | |
| b | 20 ppm Aspergillus | 1280 | 1253 | 367 | 370 |
| | glucose oxidase* | | | | |

*Aspergillus glucose oxidase: Bakezyme GO10.000 BG, DSM Food Specialties, the Netherlands.

These results show that the loaf volume of the loaf made using Penicillium glucose oxidase is larger than the volume of the Control. Due to the lack of ascorbic acid in the whole formulation, the addition of ADA results in a lower volume compared to the control.

The results on hardness day 3 show that the hardness of the loafs made using ADA and of loafs made using Penicillium glucose oxidase has decreased. Decrease in hardness is an indication for improved crumb structure.

These results illustrate that ADA in baking processes can successfully be replaced by a Penicillium glucose oxidase.

The dough containing Penicillium glucose oxidase has an increased volume and a lower hardness than the Control and thus showed an improved shock resistance as compared to the Control.

The dough containing Penicillium glucose oxidase has an increased volume and a lower hardness than the chemical oxidizer ADA and thus showed an improved shock resistance as compared to the chemical oxidizer ADA.

### Example 4

A liquid sponge also known as flour brew or liquid flour ferment was prepared via the following steps:
Add total water at 25° C. to the fermentation tank (beaker 2000 ml)
Add the flour, dry ingredients and instant dry yeast.
Mix all ingredients with a hand whisker
Start agitation (330 rpm)
keep fermentation under agitation (330 rpm) for 4 hours.

Room temperature in the lab was 22° C.

For agitation of the liquid sponges mixers were used. (IKA EUROSTAR—power control visc stirrer; Speed: 330 rpm using a stainless steel stir of 3 cm (1.18 inch) diameter and beakers of 2000 ml.)

The following recipes were used:

TABLE 7
liquid sponge recipes

| Ingredients liquid sponge | | | |
|---|---|---|---|
| recipes* | ADA | | Test |
| King Arthur Flour (USA) | 500 | g | 500 g |
| Water | 670 | g | 610 g |
| Bakeyme P 500 | 10 | ml | 10 ml; this |
| (concentration: 1 g/l) | | | corresponds to 10 |
| | | | ppm based on total |
| | | | flour* i.e. 1000 gram |
| Penicillium glucose oxidase | | | 40 ml |
| (concentration: 1 g/l) | | | (concentration: 1 g/l); |
| | | | this corresponds to 40 |
| | | | ppm based on total |
| | | | flour (1000 g) |
| Bakezyme AG 800 | | | 10 ml; this |
| (concentration: 1 g/l) | | | corresponds to 10 |
| | | | ppm based on total |
| | | | flour (1000 g) |
| ADA (25% purity) | 0.13043 | g | |
| Salt | 3 | g | 3 g |
| Sugar | 3 | g | 3 g |
| Ascorbic Acid (concentration: | | | 20 ml; this |
| 1 g/l) | | | corresponds to 20 |
| | | | ppm based on total |
| | | | flour* (1000 g) |
| Yeast (instant dry) | 10 | g | 10 g |

*Note after preparing the liquid sponge, in the dough stage among others 500 gram of flour is used making the weight of the total amount of flour used in the recipe 1000 gram (see Table 8 for further ingredients used in the dough stage). From the doughs bread was baked (details below) and evaluated by an experienced baker and judged as good.

TABLE 8
Dough recipe

| Ingredients dough recipes | ADA | Test |
|---|---|---|
| King Arthur Flour (USA) | 500 g | 500 g |
| Water | 20 g | 20 g |
| Yeast instant dry | 7.5 g | 7.5 g |
| Sugar | 70.0 g | 70.0 g |
| Shortening | 40.0 g | 40.0 g |
| Salt | 20.0 g | 20.0 g |
| Calcium Propionate | 3.2 g | 3.2 g |

Viscosity of the sponge was measured using a viscometer (Brookfield-DV-II+PRO) for viscosity measurements every 30 min at two different speeds: 50 and 100 rpm.

The results of the viscosity measurements is shown below in Tables 9 and 10.

TABLE 9
Viscosity of the liquid sponges (cPs (torque))
using 50 rotations per minute (50 RPM).

| Time (minutes) | ADA cPs (torque) | Test cPs (torque) |
|---|---|---|
| 30 | 5840 | 5120 |
| 60 | 3400 | 2350 |
| 90 | 2400 | 2296 |
| 120 | 2240 | 2168 |
| 150 | 2300 | 2312 |
| 180 | 2336 | 2410 |
| 210 | 2352 | 2256 |
| 240 | 2432 | 2432 |

TABLE 10
Viscosity of the liquid sponges using
100 rotations per minute (100 RPM).

| Time (minutes) | ADA cPs (torque) | Test cPs (torque) |
|---|---|---|
| 30 | 3000 | 2600 |
| 60 | 2600 | 1900 |
| 90 | 2012 | 1796 |
| 120 | 1812 | 1704 |
| 150 | 1836 | 1784 |
| 180 | 1808 | 1868 |
| 210 | 1868 | 1812 |
| 240 | 1872 | 1864 |

Test: 40 ppm Penicillium glucose oxidase+10 ppm of Bakezyme AG 800 (Fungal Amyloglucosidase from DSM)+20 ppm Ascorbic Acid.

This shows the combination of Penicillium glucose oxidase, Amyloglucosidase and Ascorbic Acid is suitable to replace ADA in a liquid Sponge.

From the sponges a dough was prepared as follows using the ingredients listed in Table 8.

Ingredients of the dough were put together with the liquid sponge in a Hobart (vertical) mixer and mixed for 2 minutes in (1^{st} speed) low speed and 8 minutes in (2^{nd} speed) medium speed.

After mixing the dough was rested for 3 minutes. The bread dough was divided into equal pieces of 620 g. The pieces were rested 2 minutes before molding. The pieces were then molded and placed in bread pans (4 strap Pullman bread—Chicago Metallic—top dimensions 4 in×13 in (10.16 cm×33.02 cm), bottom dimensions 3.75 in×12.75 in (9.525 cm×32.385 cm), height 4 in (10.16 cm). The bread pans were placed in the proof box for 60 minutes fermentation at 38° C. and 85% relative humidity. After fermentation, the bread pans were placed in the oven at 220° C. for 22 minutes. The breads were evaluated by an experienced baker and judged as good.

1. A product comprising a composition comprising a Penicillium glucose oxidase, said product being a chemical oxidizer replacement in the baking industry.

2. A product according to claim 1, wherein the composition further comprises ascorbic acid.

3. A product according to claim 1, wherein the composition further comprises at least one of amyloglycosidase, bacterial or fungal hemicellulase and bacterial or fungal alpha amylase.

4. A product according to claim 1, wherein the glucose oxidase has an amino acid sequence having at least 75% identity to amino acids 19-604 of SEQ ID No. 1.

5. A product according to claim 1, wherein the glucose oxidase completely or partially replaces bromate.

6. A product according to claim 1, wherein the glucose oxidase completely or partially replaces azodicarbonamide (ADA).

7. A pre-mix comprising
a) a glucose oxidase with an amino acid sequence having at least 75% identity to amino acids 19-604 of SEQ ID No.1;
b) 0 to 20 ppm azodicarbonamide based on flour; and
c) 0 to 30 ppm bromate based on flour.

8. A method for improving flour, which method comprises adding to the flour by weight of flour 1 to 50 ppm of a Penicillium glucose oxidase having an activity in the range of 2500-4000 glucose oxidase units/gram enzyme.

9. A flour comprising by weight of flour
a) 1-50 ppm of a glucose oxidase with an amino acid sequence having at least 75% identity to amino acids 19-604 of SEQ ID No.1;
b) 0 to 20 ppm azodicarbonamide; and
c) 0 to 30 ppm bromate.

10. A flour according to claim 8 comprising by weight of flour
a) 1-50 ppm of a glucose oxidase with an amino acid sequence having at least 75% identity to amino acids 19-604 of SEQ ID No.1;
b) 10-50 ppm ascorbic acid;
c) 0-20 ppm amyloglucosidase;
d) 0 to 20 ppm azodicarbonamide; and
e) 0 to 30 ppm bromate.

11. A flour according to claim 8 comprising by weight of flour
a) 1-50 ppm of a glucose oxidase with an amino acid sequence having at least 75% identity to amino acids 19-604 of SEQ ID No.1;
b) 10-50 ppm ascorbic acid;
c) 1-20 ppm fungal alpha amylase;
d) 1-50 ppm hemicellulase;
e) 0 to 20 ppm azodicarbonamide; and
f) 0 to 30 ppm bromate.

12. A method for preparing a dough comprising combining a pre-mix according to claim 7 with at least one dough ingredient.

13. A dough comprising a Penicillium glucose oxidase, said glucose oxidase comprising an amino acid sequence having at least 75% identity to amino acids 19-604 of SEQ ID No. 1 and one or more dough ingredients.

14. A method for preparing a baked product comprising the step of baking the dough according to claim 13.

15. A baked product prepared from a pre-mix according to claim 7 or prepared from a dough comprising a Penicillium glucose oxidase, said glucose oxidase comprising an amino acid sequence having at least 75% identity to amino acids 19-604 of SEQ ID No. 1 and one or more dough ingredients.

16. A method to replace a chemical oxidiser in a baking product comprising adding Penicillium glucose oxidase, as set out in amino acids 19-604 of SEQ ID No.1, to at least one dough ingredient.

17. A method according to claim 16, wherein the Penicillium glucose oxidase is part of a composition comprising a dough ingredient and/or an additional enzyme.

18. The product according to claim 1, wherein said composition is used to stabilize a liquid sponge in the production of a baked product.


SEQUENCE LISTING (2 sequences)

SEQ ID NO: 1 - protein, 604 aa, Penicillium chrysogenum
  SIGNAL (1)..(18)
MKSTIITSILFSVAAVQAYSPAEQIDVQSHLLSDPTKVEGETYDYVIAGGGLTGLTVAAKLSENPKIKVLVIEKGFYESNDGPIIEDPNAYGEIFGTSVDQNYLTVPLINNRTGEIKSGLGLGGSTLINGDSWTRPDKVQIDSWEKVFGMEGWNWDNVFQYMQKAERSRPPTAAQIEAGHFYDPACHGTDGTVHAGPRDNGKPWSPLMRALMNTVSAFGVPVQKDFHCGHPRGVSMIPNNLHENQIRADAAREWLLPNYQRDNLQILTGQKVGKVLFNQTASGPKAVGVNFGTNKAVNFNVYAKQEVLLAAGSAISPLILEYSGIGIKSVLDKAGVKQLLELPVGLNMQDQTTTTVRSRANNAPGQGQAAYFANFTEVLGDHAAQGIKLLDTKLDQWAEETVARGGFHNVTALKIQYENYRNWLLDEDVAFAELFFDTEGKINFDIWNLIPFTRGSVHILSSDPYLWQYANDPKFFMNELDLLGQAAATKLGRELSSAGEMKKYYAGETIPGDNLPQDATVEQWEDYVMMNFRPNWHAVSTCSMMSRELGGVVDATAKVYGTQGLRVIDGSIPPTQVSSHVMTVFYGMALRIAESVLEDYAKKA

SEQ ID NO: 2 - DNA, 1816 bp, Penicillium chrysogenum
  sig_peptide (1)..(54)
  terminator (1813)..(1816)
atgaagtccactattatcacctccattctcttctctgtggctgccgtccaggcctatagcccggccgagcagatcgacgtccagtctcacctgctttctgaccccaccaaggtcgagggagagacttacgactatgtcattgctggtggtggtttgactggtctgaccgtggctgccaagctgtctgaaaacccgaagatcaaagtccttgtgattgagaagggattctacgaatccaacgatggaccgatcatcgaggaccccaacgcctatggggagatctttggaactagtgtggatcagaattatctcacagttcccctcatcaacaaccgaactggggaaattaagtctggcctcggtcttggtggctcgaccttgatcaacggcgattcctggacccgccccgacaaggtccagatcgactcatgggaaaaggtctttggcatggagggctggaactgggacaatgtcttccagtacatgcagaaagctgagcgctcgcgccccccgactgccgcccagattgaagccggtcacttctacgaccctgcctgtcatggaacagacggaaccgttcatgccggccctcgcgacaacggcaagccttggtccccactgatgcgagccctcatgaacaccgtctccgctttcggtgtccccgtccagaaggacttccactgcggtcacccccgtggtgtctcgatgatcccgaacaacctccatgagaaccagatccgggctgatgccgctcgcgaatggcttcttcccaactaccagcgcgataacctgcagatcctgactggccagaaggtcggaaaggttttgttcaaccagaccgcatctggacctaaggctgttggtgtgaacttcggtaccaacaaggctgttaacttcaatgtctacgccaagcaagaagttctgttggccgccggatctgccatttctcctttgatccttgaatactccggtattggtatcaagtccgtccttgacaaggccggtgttaagcagctcctcgaactccctgttggtctcaacatgcaagaccagaccactaccactgttcggtcccgcgccaacaacgcacctggacaaggccaggccgcttactttgccaacttcaccgaggttctcggcgaccacgccgcccagggtattaagttgctggacaccaagcttgaccagtgggccgaggagaccgttgcccgcggtggcttccacaatgtgactgccctcaagatccagtatgagaactaccgtaactggctccttgatgaggacgttgcatttgccgagctcttcttcgacactgagggcaagatcaactttgatatctggaatcttatccccttcactcgcggttccgtccacatcctcagcagtgacccttacctctggcaatacgcaaatgaccccaagttcttcatgaacgagctggatcttctcggccaggccgctgctactaagctgggtcgtgagctctctagcgctggtgagatgaagaagtactacgctggcgagaccatccccggcgacaacctgccccaggatgccaccgtcgagcagtgggaggactacgtgatgatgaacttccgtcctaactggcacgctgttagcacctgctctatgatgtcccgcgagcttggtggtgtcgtcgacgctactgccaaggtctacggtactcagggcctccgtgtcattgacggatctatccctcccactcaggtgtcctctcacgttatgaccgttttctacggtatggccttgcggatcgccgaatccgtccttgaagactatgccaagaaagcttaaa